SEQUENCE LISTING (1 sequence)

SEQ ID NO: 1 - RNA, 20 nt, Artificial sequence
  synthesized
ggggccacuagggacaggau

(19) United States
(12) Patent Application Publication
(10) Pub. No.: US 2023/0383248 A1
(43) Pub. Date: Nov. 30, 2023

(54) METHOD FOR OBTAINING GLIAL CELLS IN VITRO AND USE THEREOF

(71) Applicant: XELLSMART BIOMEDICAL (SUZHOU) CO., LTD., Shanghai (CN)

(72) Inventor: Xiang LI, Shanghai (CN)

(21) Appl. No.: 18/032,592

(22) PCT Filed: Oct. 19, 2021
(86) PCT No.: PCT/CN2021/124649

§ 371 Date: Apr. 19, 2023

(30) Foreign Application Priority Data
Oct. 20, 2020 (CN) 202011128104.2

(51) Int. Cl. C12N 5/079; A61K 35/30

(52) U.S. Cl. CPC C12N 5/0622; A61K 35/30; C12N 2501/15; C12N 2501/155; C12N 2501/11; C12N 2501/115; C12N 2501/235; C12N 2506/02; C12N 2506/45; C12N 2510/00; C12N 2503/02

(57) ABSTRACT

The present disclosure provides a method for obtaining glial cells in vitro and use thereof. The method comprises: constructing positive cloned stem cells that overexpress a reprogramming factor, wherein the reprogramming factor comprises an NFIX gene; and inducing the positive cloned stem cells to the glial cells by adding a cytokine and/or a cytokine inhibitor. The method can rapidly induce the pluripotent stem cells to differentiate into the glial cells, and the obtained glial cells can be used for preparing cell treatment drugs and in-vitro or in-vivo drug screening kits.

### CROSS-REFERENCE TO RELATED APPLICATION

This present disclosure is a national stage filing under 35 U.S.C. § 371 of international application number PCT/CN2021/124649, filed Oct. 19, 2021, which claims priority to Chinese patent application No. 2020111281042, filed on Oct. 20, 2020. The contents of the international application are incorporated herein by reference in its entirety.

### INCORPORATION BY REFERENCE

This application includes a sequence listing in computer readable form (a “txt” file) that is submitted herewith on ASCII text file named P23JM1NW00043US_SEQUENCE_LISTING.txt, created on Apr. 19, 2023 and 465 bytes in size. This sequence listing is incorporated by reference herein.

### TECHNICAL FIELD

The present disclosure relates to the technical field of cell biology, particularly to a method for obtaining glial cells in vitro and use thereof.

### BACKGROUND

At present, in the obtaining of functional cell types via cell fate reprogramming and induced differentiation, how to achieve more precise cell fate regulation and subtype specialization is of great significance for the final application of the obtained functional cell types and understanding of the mechanism of cell fate determination. Especially, in the induction and differentiation of nerve cell fate, precise preparation of different types of functional subtype nerve cells, and deep understanding of the relationship between the functional subtype nerve cells and the occurrence and development of nervous system diseases are keys to the ultimate realization of cell therapy for nervous system diseases. Recent studies have proved that the functional loss or damage of glial cells is closely related to the occurrence and development of a series of nervous system diseases, and different brain and/or spinal cord specialized subtype glial cells play different roles in a central nervous system.

There are many differences between non-primate (such as mice and dogs) cells and primate (such as humans) cells in the aspects of property and functionality. A disease model for drug development established by non-primate (such as mice and dogs) cells has obvious defects. However, primate cells such as human cells have the problem of a severely limited source. A directed induced differentiation method based on human pluripotent stem cells is used to prepare functional cell types, which makes it possible to study the characteristics of different human cell types in vitro and their relationship with occurrence and development of diseases, and establish a disease model in a cell level for the development of new drugs.

The methods for directed induced differentiation of pluripotent stem cells into glial cells in the prior art mainly have the following defects.
(1): in the traditional method, an induction method is conducted in stepwise to simulate the development in vivo, which takes a long time. By taking the glial cells (mainly astrocytes) as an example, the traditional directed induced differentiation method lasts for 3-6 months, which limits the researches based on the induced differentiated glial cells.
(2): the function of the glial cells obtained by induction is not strictly demonstrated.
(3): the induction efficiency and maturity of the obtained glial cells are low, and need to be further improved.

Based on the above reasons, it is needed to further study on methods for directed induced differentiation of pluripotent stem cells into the glial cells, so as to solve the problem that directed induced differentiation for obtaining brain and/or spinal cord specialized subtype glial cells cannot be rapidly performed.

### SUMMARY

The main objective of the present disclosure is to provide a method for obtaining glial cells in vitro and use thereof to solve the problem in the prior art that directed induced differentiation for obtaining glial cells cannot be rapidly performed, and further solve the problem of obtaining brain and/or spinal cord specialized subtype glial cells by induction of glial cells.

To achieve the above objective, according to one aspect of the present application, provided is a method for obtaining glial cells in vitro, comprising:
1) constructing positive cloned stem cells that overexpress a reprogramming factor, wherein the reprogramming factor comprises an NFIX gene; and
2) inducing the positive cloned stem cells into the glial cells by adding a cytokine and/or a cytokine inhibitor.

In some embodiments, the positive cloned stem cells are constructed by a conventional gene overexpression method in the art.

In some embodiments, the positive cloned stem cells are constructed by a CRISPR/Cas9 system.

In some embodiments, the stem cells are primate stem cells or non-primate mammal stem cells.

In some embodiments, the primate stem cells are human stem cells or non-human primate stem cells.

In some embodiments, the stem cells are pluripotent stem cells or neural stem cells.

In some embodiments, the pluripotent stem cells are embryonic stem cells and/or induced pluripotent stem cells.

In some embodiments, the human stem cells are human pluripotent stem cells or human neural stem cells.

In some embodiments, the human pluripotent stem cells are human embryonic stem cells and/or human induced pluripotent stem cells.

In some embodiments, the human embryonic stem cells are commercial human embryonic stem cells.

In some embodiments, the human embryonic stem cells are stem cells isolated or obtained from human embryos which are not developed in vivo within 14 days of fertilization.

In some embodiments, the reprogramming factor further comprises at least one of other genes which are beneficial for directed differentiation into glial cells.

In some embodiments, the other genes which are beneficial for directed differentiation into glial cells are at least one genes from NFI family genes. In some embodiments, the other genes which are beneficial for directed differentiation into glial cells are at least one of other nuclear factor genes in NFI family genes excluding NFIX. In some embodiments, the other nuclear factor genes are NFIA and/or NFIB.

In some embodiments, the reprogramming factor is an NFIX gene.

In some embodiments, the obtained glial cells are one or more selected from the group consisting of astrocytes, oligodendrocytes, microglia and other types of glial cells. In some embodiments, the glial cells are astrocytes.

In some embodiments, the cytokine and/or the cytokine inhibitor in the step 2) is one or more of: an ectodermal and neural differentiation promoting factor and/or a non-neural differentiation promoting inhibitor, a neural differentiation promoting factor, or a glial cell maturation promoting factor.

In some embodiments, in the step 2), the inducing the positive cloned human stem cells into the glial cells by adding the cytokine and/or the cytokine inhibitor includes three-stage cultivation. In some embodiments, the ectodermal and neural differentiation promoting factor and/or the non-neural differentiation inhibitor is added in the first stage, the neural differentiation promoting factor is added in the second stage, and the glial cell maturation promoting factor is added in the third stage.

In some embodiments, the ectodermal and neural differentiation promoting factor and/or the non-neural differentiation promoting inhibitor are/is a transforming growth factor inhibitor. In some embodiments, the transforming growth factor inhibitor is a TGF-β inhibitor and/or a BMP inhibitor.

In some embodiments, the neural differentiation promoting factor is an exogenous activator. In some embodiments, the exogenous activator is at least one of: a fibroblast growth factor, an epidermal growth factor, or a small molecule functional analogue and/or other functional analogues.

In some embodiments, the glial cell maturation promoting factor is one or more of: a leukocyte inhibitory factor, a fetal bovine serum, a newborn bovine serum, an adult bovine serum and sheep serum and their analogues, a BMP activator, a neurotrophic factor, and/or other reported glial cell maturation promoting factors.

In some embodiments, provided is a method for obtaining a brain and/or spinal cord specialized subtype glial cells in vitro, on the basis of the above steps 1) and 2), further comprising a step 3): inducing the glial cells to brain and/or spinal cord specialized subtype glial cells by adding at least one of other cytokines and/or cytokine inducers.

In some embodiments, the brain and/or spinal cord specialized subtype glial cell is one or more selected from the group consisting of: forebrain specialized subtype glial cells, midbrain specialized subtype glial cells, back brain specialized subtype glial cells, and different spinal cord segment specialized subtype glial cells.

According to another aspect of the present disclosure, provided are glial cells obtained by the above method.

In some embodiments, provided are brain and/or spinal cord specialized subtype glial cells obtained by the above method.

According to another aspect of the present disclosure, provided is a drug, comprising the glial cells obtained by the above method.

According to another aspect of the present disclosure, provided is a drug, comprising a) the glial cells obtained by the above method; and/or b) the brain and/or spinal cord specialized subtype glial cells obtained by the above method.

In some embodiments, the drug is a cell treatment drug.

In some embodiments, the drug further comprises at least one pharmaceutically acceptable carrier.

According to another aspect of the present application, provided is use of the above glial cells in the preparation of a drug for preventing and/or treating a nervous system disease.

In some embodiments, provided is use of a) glial cells and/or b) brain and/or spinal cord specialized subtype glial cells in the preparation of a drug for preventing and/or treating a nervous system disease.

In some embodiments, the nervous system disease is a neurodegenerative disease. The non-limiting examples of the neurodegenerative diseases include Alzheimer's disease, amyotrophic lateral sclerosis, Parkinson's disease, schizophrenia, glioblastoma, Huntington's disease, multiple sclerosis, and the like.

According to another aspect of the present disclosure, provided is an in-vitro or in-vivo drug screening kit, comprising the glial cells obtained by the above method.

In some embodiments, provided is an in-vitro or in-vivo drug screening kit, comprising a) the glial cells obtained by the above method; and/or b) the brain and/or spinal cord specialized subtype glial cells obtained by the above method.

According to another aspect of the present disclosure, provided is a kit for inducing the differentiation of human stem cells, comprising:
1) at least one reprogramming factor overexpression reagent, wherein the reprogramming factor comprises an NFIX gene; and
2) at least one cytokine and/or cytokine inhibitor for inducing the positive cloned stem cells into the glial cells.

In some embodiments, the kit further comprises: 3) at least one of other cytokines and/or cytokine inducers for further inducing the glial cells into the brain and/or spinal cord specialized subtype glial cells.

By using the technical solution of the present disclosure, it can rapidly induce the differentiation of human pluripotent stem cells into glial cells, with high induction efficiency and good induction maturity; further, the problem that directed induced differentiation for obtaining brain and/or spinal cord specialized subtype glial cells cannot be rapidly performed can be solved. The obtained glial cells can be used for preparing cell treatment drugs and in-vitro or in-vivo drug screening kits.

### BRIEF DESCRIPTION OF DRAWINGS

To more clearly illustrate the technical solution of the embodiments of the present application, drawings required to be used in the description of the embodiments will be simply introduced, obviously, the drawings in the following description are only some embodiments of the present application. Other drawings can also be obtained by persons of ordinary skill in the art according to these drawings, which does not go beyond the protective scope of the present application.

FIG. 1 is a graph showing the cell fluorescence (GFAP) display results of Example 1, Comparative example 1, Comparative example 2 and Comparative example 3. Where, a) is a non-induction group, b) is a non-NFI family gene induction group, c) is an NFIA rapid induction group, and d) is an NFIX rapid induction group.

FIG. 2 is a bar graph showing the cell fluorescence display results of Example 1, Comparative example 1, Comparative example 2 and Comparative example 3.

FIG. 3 is a bar graph showing the neurite quantity comparison results of Example 1, Comparative example 2 and Comparative example 3.

FIG. 4 is an identification graph of glial cells obtained in Example 1, where a) is Ho staining, b) is S100 β staining, c) is GFAP staining, and d) is a combination of three staining methods.

FIG. 5 is a graph showing the fluorescence display result of co-culture of exogenous GFP strong-expression in-vivo labeled neuron cells and NFIX rapid induced glial cells in Example 7, where a) is control group, with neurons alone, and b) is experimental group, i.e., co-culture of glial cells obtained by induction in Example 1 and neuron cells.

### DETAILED DESCRIPTION

The technical solution in embodiments of the present application will be clearly and completely described in combination with drawings in embodiments of the present application, obviously, the described embodiments are some embodiments of the present application but not all the embodiments. Based on the embodiments of the present application, other embodiments obtained by those skilled in the art without creative efforts all fall within the protective scope of the present application.

It is understood that the embodiments of the present disclosure and features of the embodiments can be mutually combined without conflict. Next, the present disclosure will be illustrated in detail in combination with embodiments.

The present disclosure will be further described in detail in combination with specific embodiments, and these embodiments cannot be understood as limiting the protective scope of the present application.

1. Method for Obtaining Glial Cells In Vitro

As described in the background, the existing methods for directed inducing pluripotent stem cells to differentiate into glial cells have the problems that directed induced differentiation for obtaining the glial cells cannot be rapidly performed, and cannot further obtain the brain and spinal cord specialized subtype glial cells. To solve the above problems, the present disclosure provides a method for obtaining glial cells in vitro, comprising:
1) constructing positive cloned stem cells that overexpress a reprogramming factor, wherein the reprogramming factor comprises an NFIX gene; and
2) inducing the positive cloned stem cells into the glial cells by adding a cytokine and/or a cytokine inhibitor.

The glial cells play a key role in maintaining a normal central nervous system function, and the lesion of subtype specialized glial cells is closely related to the occurrence and development of a series of nervous system diseases. Based on a directed induced differentiation method for pluripotent stem cells, glial cells, especially subtype specialized glial cells may be prepared efficiently in-vitro, which has an important research and application value. The method for obtaining glial cells in vitro provided by the present disclosure combines the idea and method of cell fate reprogramming with the induced differentiation of pluripotent stem cells. By taking astrocytes and human stem cells as examples, starting from glial cell fate determinants during the development, several human pluripotent stem cell lines are constructed for induced expression of different glial cell fate determinants, and finally a rapid and direct induction method of astrocytes based on the NFI family is established (3-6 months of induction cycle required in the prior art is shortened to 4-8 weeks) and the properties of the obtained glial cells are demonstrated. Furthermore, by conducting subtype specialization induction on different brain regions and/or spinal cord regions during the induction, a rapid differentiation method for obtaining brain and/or spinal cord specialized subtype astrocytes can be further established.

Both of oligodendrocytes and astrocytes come from neural stem cells of an early central nervous system in the processes of embryonic development and in-vitro induced differentiation. The neural stem cells can differentiate into neurons, astrocytes and oligodendrocytes. The NFI family plays an important role in the critical fate determination process of embryonic development and in-vitro induced differentiation of astrocytes, oligodendrocytes and microglia. For example, in Benjamin Deneen et al. “The Transcription Factor NFIA Controls the Onset”, it is mentioned that: we identified a family of transcription factors, called NFI genes, which are induced throughout the spinal cord ventricular zone (VZ) concomitantly with the induction of GLAST, an early marker of gliogenesis; NFIA is also essential for the continued inhibition of neurogenesis in VZ progenitors; NFIA links the abrogation of neurogenesis to a generic program of gliogenesis, in both astrocyte and oligodendrocyte VZ progenitors. In Yong Wee Wong et al. “Gene expression analysis of nuclear factor I-A deficient mice indicates delayed brain maturation”, it is mentioned that: in the early postnatal period, brain development, especially oligodendrocyte maturation, is delayed in NFIA −/−mice, and the marker genes for differentiating neural cells are downregulated. However, the inventor unexpectedly discovered that overexpressing the NFIX gene is more effective than overexpressing the NFIA gene recognized by those skilled in the art to promote the development of glial cells. The NFI family genes play a key role in the differentiation and specialization of different parts and different types of glial cells in the central and peripheral nervous systems in terms of neural development. The method of the present disclosure can also be reasonably used in the induction of different types of glial cells, including astrocytes, oligodendrocytes, microglia and other types of glial cells.

1.1 Gene Overexpression

In addition, the conventional gene overexpression methods in the art are all applicable to this method, and all gene overexpression methods are interchangeable. Gene overexpression methods can at least include but are not limited to the following methods:
1) viral vector-mediated integrated or non-integrated gene overexpression;
2) overexpression in which the mRNA of a target gene is introduced;
3) overexpression in which a protein of a target gene is introduced;
4) overexpression that is achieved by CRISPR/Cas9 or other gene editing tools;
5) overexpression that is achieved through small molecules, microRNA (for example microRNA-153), or other methods that can activate endogenous expression of target genes;
6) Methods for achieving the overexpression of the target gene by targeting endogenous inhibitory factors to relieve the inhibition effect of the target factor by means of above 5 methods.

Conventional gene overexpression methods can be seen in the following references:
https://www.nature.com/articles/nbt.3070;
https://www.sciencedirect.com/science/article/pii/S2213671115001873; and
https://www.nature.com/articles/nrg2937.

In some embodiments, the positive cloned stem cells are constructed by a clustered regularly interspaced short palindromic repeats (CRISPR)/Cas9 system. The CRISPR/Cas9 gene editing technology is a technology that is used for specific DNA modification of a target gene, and the gene editing technology based on CRISPR/Cas9 has significant application prospect in the application fields of a series of gene therapies, such as hematological diseases, tumors, and other genetic diseases. These technological achievements have been applied to precise genome modification of human cells, zebrafish, mice, and bacteria.

The CRISPR/Cas9 gene editing technology is known as one of the biggest biotechnology discoveries in this century, and its inventor won the Nobel Prize in Chemistry in 2020. The CRISPR/Cas9 gene editing technology has been sufficiently proved that it is capable of realizing fixed-point editing and modification (insertion, knockout, mutation, etc.) of target genes in-vivo or in-vitro.

In a human pluripotent stem cell line, it has been fully demonstrated that a method of inserting the sequence of the gene to be overexpressed into a safe integration site (such as AAVS1) of a genome based on the CRISPR/Cas9 technology can achieve stable and efficient overexpression of genes. Compared to the alternative gene overexpression methods such as randomly integrated virus transfection, such the method has a safer and more stable advantage.

The NFIX gene overexpression method based on the CRISPR/Cas9 technology specifically comprises the following steps: an NFIX CDS sequence (Gene ID: 4784; NM_001271043.2) is obtained from NCBI website, a sequence comprising NFIX CDS with two end enzyme digestion site sequences of SalI (GTCGAC) and MluI (ACGCGT) is obtained through full synthesis, the NFIX CDS-SalI-MluI sequence is enzyme-digested, recovered and connected to the framework plasmid of AAVS1-TRE3G-SalI-MluI, and then transfected into the human pluripotent stem cells by the reported methods and transfection procedures, and clones are picked for subsequent passage. The selected gRNA target sequence is GGGGCCACTAGGGACAGGAT (Addgene #41818; http://n2t.net/addgene:41818;RRID:Addgene_41818).

The gene overexpression method based on the CRISPR/Cas9 technology can be seen in the following references:
1. Qian, K., et al. (2014). A simple and efficient system for regulating gene expression in human pluripotent stem cells and derivatives. Stem Cells 32, 1230-1238.
2. Li, X., et al., and Zhang, S C. (2018). Rapidly Generation of Functional Subtype Astrocytes from Human Pluripotent Stem Cells. Stem Cell Reports 11, 998-1008.

1.2 Stem Cells

Stem cells are a class of cells having an infinite or eternal self-renewal ability, which can generate at least one type of highly differentiated offspring cells. The present disclosure can be implemented using various types of stem cells. The stem cells can be derived from different sources, and the non-limiting examples of the sources include primates (such as humans or non-human primates) or non-primate mammals. The non-limiting examples of the stem cells include omnipotent stem cells, pluripotent stem cells, induced pluripotent stem cells, and unipotent stem cell. In some embodiments, the stem cells can be the pluripotent stem cells (for example, induced pluripotent stem cells and embryonic stem cells) or neural stem cells.

The non-limiting examples of human stem cells include human embryonic stem cells, human pluripotent stem cells, human induced pluripotent stem cells, human neural stem cells, human parthenogenetic stem cells, human primitive germ cell like pluripotent stem cells, human ectoderm stem cells, human F-class pluripotent stem cells, human adult stem cells, human cancer stem cells or any other cells capable of lineage differentiating. In some embodiments, the human pluripotent stem cells (PSCs) are human embryonic stem cells (hESCs) (such as H1 and H9) and/or human induced pluripotent stem cells (hiPSCs) (such as WC50 and IMR90). The human pluripotent stem cells are a class of pluripotent cells having self-renewal and self-replication abilities. The pluripotent stem cells have a potential to differentiate into various cell tissues, but lose the ability to develop into complete individuals, whose development potential is limited in a certain extent. In the practical application, the human pluripotent stem cells may be replaced with other human stem cells or other human somatic cells according to the actual situation. In some embodiments, the human embryonic stem cells are commercial human embryonic stem cells. In some embodiments, the human embryonic stem cells are stem cells isolated or obtained from human embryos which are not developed in vivo within 14 days of fertilization.

The emerging cell therapy is a new hope in the field of regenerative medicine. At present, there are two bottlenecks: it is difficult to obtain high-quality seed cells and sufficient number of cells; and it is difficult to expand infinitely. The proliferation ability of adult stem cells from specific tissue sources is limited, and the number of cells that can be obtained each time is limited, making it difficult to achieve large-scale application. However, repeated sampling and preparation can significantly increase production cost. The starting point of constructing recombinant cells in the induced differentiation method of the present disclosure may be pluripotent stem cells with full pluripotency, which can differentiate into more than 200 cell types and subtypes of three human embryo layers. As is well known, the lower the degree of differentiation of stem cells, the higher the complexity of their regulation. The inventor, through extensive preliminary researches on numerous key regulatory genes during the differentiation process, ultimately obtained a NFIX gene induced rapid differentiation scheme for induction of human pluripotent stem cells into glial cells. Based on the advantage of unlimited proliferation of human pluripotent stem cells, a large number of high-quality glial cells can be prepared through rapid induction. In practical applications, seed cells can be amplified first according to the needs and objectives of industrialization to obtain the required number of cells, which has significant advantages in expanding industrial production while reducing time and process costs and increasing batch stability.

In some embodiments, the stem cells are neural stem cells (for example, neural stem cells). The neural stem cell (NSC) is a cell population that is present in a nervous system, has a potential to differentiate into neurons and various classes of glial cells so as to generate a large amount of brain cell tissues, can self-renew and can sufficiently provide a large number of brain tissue cells. Compared with the pluripotent stem cells, the neural stem cells have higher differentiation degree, and the difficulty of regulating their induction into glial cells is lower. Those skilled in the art can refer to the following solution to reduce the difficulty of induction, for example Jason Tchieu et al, NFIA is a gliogenic switch enabling rapid derivation of functional human astrocytes from pluripotent stem cells. Those skilled in the art can also refer to the following document concerning NFIA genes, Rapidly Generation of Functional Subtype Astrocytes from Human Pluripotent Stem Cells, Stem Cell Reports 11, 998-1008, had been publicly reported by the inventor, embryonic stem cells were induced into neural stem cells, and then the neural stem cells were further induced into astrocytes through reprogramming technology. Therefore, the NFIX rapid induction scheme of the present disclosure is also applicable to an induction process starting from neural stem cells.

1.3 NFIX Genes and their Family Genes

The method of the present disclosure comprises: constructing positive cloned human stem cells that overexpress a reprogramming factor, wherein the reprogramming factor comprises an NFIX gene. In some embodiments, the reprogramming factor further comprises at least one of other genes which are beneficial for directed differentiation into glial cells. The non-limiting examples of other genes which are beneficial for directed differentiation into glial cells include NFIA, Nkx gene family (such as Nkx6.2, Nkx2.1), Olig gene family (such as Olig1 and Olig2), PAX, SOX gene family (such as SOX2, SOX1, SOX9 and SOX10), HOXA family genes, and HOXB family genes. In some embodiments, the other genes which are beneficial for directed differentiation into glial cells are at least one genes from NFI family genes. The non-limiting examples of NFI family genes include NFIA, NFIB and NFIC. In some embodiments, the other genes which are beneficial for directed differentiation into glial cells are at least one of other nuclear factor genes in NFI family genes excluding NFIX; that is, the reprogramming factor is a combination of NFIX genes and other nuclear factor genes excluding NFIX. In some embodiments, the other nuclear factor gene are NFIA and/or NFIB.

All of NFIA, NFIB and NFIX belong to NFI transcription factor family, which can not only bind to a promoter region of a target gene but also recruit other related factors to regulate the transcription of the target gene. NFI family gene expression products are a class of proteins necessary for in vitro replication of adenovirus DNA. At present, there are many researches on NFIA and NFIB, but there are few of researches on NFIX gene function. Since 1980s, NFIX genes (nuclear factor I-X genes) have been discovered, the existing researches mainly suggest that NFIX can inhibit cell proliferation, and NFIX mutation can cause an obstructed muscle tissue metabolism. No researches suggest that NFIX can in vitro induce directed differentiation of stem cells into glial cells.

In some embodiments, the reprogramming factor is an NFIX gene. That is, expression of NFIX genes alone can rapidly induce the differentiation into glial cells. In the process of nervous development, completely differentiated functional cells undergo several intermediate stages: embryonic stem cells firstly generate an early neuroectoderm, so as to produce region specialized neural progenitor cells; the neural progenitor cells further differentiate into post-mitotic neurons which are a fully differentiated cell in brain. Even though in thus simplified description, the terminal cell fate is also determined through three consecutive cell fate transitions. Since the current researchers lack systematic understanding of a three-embryonic layer differentiation system of human pluripotent stem cells, the differentiation pathway is unclear; meanwhile, most research methods do not systematically understand the complexity and heterogeneity of cell components during the differentiation, and encounter bottlenecks in the process of further optimization, resulting in the inability to obtain the final required functional cells. Thus, most of the differentiation solutions in relevant researches of pluripotent stem cells differentiating into histiocyte on each embryo layer still have the problems of low differentiation efficiency, cellular functional defects and the like, and finally a bottleneck occurs. Through extensive preliminary researches, the inventor revealed numerous key regulatory genes (including NFIX, NFIA, and other genes) during the differentiation, deepened the understanding of the molecular pathways involved in early embryonic lineage differentiation. After the differentiation characteristics and pathways of glial cells are deeply understood through extensive researches and experiments, it is determined that which pathways and developmental pathways are necessary. Through overexpression of NFIX transcription factors, epigenetic barriers are overcome and the early development process is bypassed, and the rapid differentiation scheme for NFIX gene induction of human PSC into astrocytes is finally achieved, which greatly shortens 3-6 months required in traditional directed induction scheme to 4-8 weeks. Moreover, the final target cells account for a high proportion and have good maturity.

In the present disclosure, the inventor prove that the NFIX gene-based rapid differentiation of inducing pluripotent stem cells into glial cells, regardless of the technology that the genes are not overexpressed or the technology that the known gene NFIA is overexpressed, can significantly improve the induced differentiation efficiency of stem cells and the maturity of the nerve cells obtained by induction.

1.4 Induction Differentiate into Glial Cells by Addition of a Cytokine and/or a Cytokine Inhibitor

In some embodiments, the cytokine and/or cytokine inhibitor is one or more selected from the group consisting of: an ectodermal and neural differentiation promoting factor and/or a non-neural differentiation promoting inhibitor; a neural differentiation promoting factor; a glial cell maturation promoting factor; and other reported cytokines and/or cytokine inhibitors that can induce positive cloned stem cells to glial cells. The ectodermal and neural differentiation promoting factor and/or non-neural differentiation promoting inhibitor are/is capable of promoting differentiation to ectoderm and nerve direction, and/or is capable of inhibiting the differentiation toward the unexpected direction (namely, non-neural direction). The nerve differentiation promoting factor has the functions of promoting the differentiation of nerves and inhibiting the growth of nerve cell tumors. The glial cell maturation promoting factor is capable of promoting the maturation of glial cells, such as astrocytes. One or more (i.e., alone or a combination) of the above cytokines and/or cytokine inhibitors are capable of inducing the positive cloned stem cells to the glial cells.

In some embodiments, in the step 2), the inducing the positive cloned stem cells into the glial cells by adding the cytokine and/or cytokine inhibitor includes three-stage cultivation. The ectodermal and neural differentiation promoting factor and/or non-neural differentiation inhibitor is added in the first stage, the neural differentiation promoting factor is added in the second stage, and the glial cell maturation promoting factor is added in the third stage. In the first stage, the positive cloned stem cells are induced to neural precursor cells; in the second stage, the neural precursor cells are induced to glial precursor cells; and in the third stage, the glial precursor cells are induced to glial cells. In some embodiments, the culture time in the first stage is 3-14 days, the culture time in the second stage is 15-35 days, and the culture time in the third stage is 2-12 days. Preferably, the culture time in the first stage is 5-12 days, the culture time in the second stage is 18-32 days, and the culture time in the third stage is 4-10 days. More preferably, the culture time in the first stage is 7-10 days, the culture time in the second stage is 21-28 days, and the culture time in the third stage is 6-8 days. The following concentrations are concentrations of various cytokines and/or cytokine inhibitors in culture media.

In some embodiments, the ectodermal and neural differentiation promoting factor and/or non-neural differentiation promoting inhibitor is a transforming growth factor inhibitor. In some embodiments, the transforming growth factor inhibitor is a TGF-β inhibitor and/or a BMP inhibitor. The mechanism of action of the transforming growth factor inhibitor (such as TGF-β inhibitor) mainly includes the following aspects: 1) inhibition of expression of TGF-β and receptors thereof; 2) obstruction of binding of TGF-β to receptors; 3) interference of receptor kinase signaling. The non-limiting examples of the TGF-β inhibitors include SB431542, LDN193189, A8301 and the like. In some embodiments, the working concentration of the TGF-β inhibitor is 0.5-20 μM, preferably 1-10 μM, further preferably 1.5-2.5 μM. In some embodiments, the working concentration of the SB431542 is 0.5-20 μM, preferably 1-10 μM, further preferably 1.5-2.5 μM. The BMP inhibitor is capable of inhibiting the BMP mediated activity of Smad1, Smad5 and Smad8, and effectively inhibiting the transcription activity of I type receptor ALK2 and ALK3 of BMP. The non-limiting examples of the BMP inhibitors include LDN193189, DMH-1 and the like. In some embodiments, the working concentration of the BMP inhibitor is 10-500 nM, preferably 50-200 nM, further preferably 80-120 nM. In some embodiments, the working concentration of the LDN193189 is 10-500 nM, preferably 50-200 nM, further preferably 80-120 nM. The above working concentrations are concentrations of various cytokines and/or cytokine inhibitors in culture media.

In some embodiments, the neural differentiation promoting factor is an exogenous activator. In some embodiments, exogenous activators are fibroblast growth factors and/or epidermal growth factors and/or small molecule functional analogues and/or other functional analogues. Fibroblast growth factors (FGFs) are polypeptides composed of about 150-200 amino acids, are present in two closely related forms, that is, basic fibroblast growth factor (bFGF) and acidic fibroblast growth factor (aFGF). FGFs, as intercellular signaling molecules, play an important role in embryogenesis and differentiation, and can induce the replication of neuroectoderm. In some embodiments, the working concentration of the fibroblast growth factor is 1-500 ng/ml, preferably 5-100 ng/ml, further preferably 10-30 ng/ml. The epidermal growth factor (EGF) can promote not only the growth of neural stem cells but also their differentiation into neurons and glial cells. In the formation of an embryonic neural tube, the expression of the epidermal growth factor can be detected in both the neuroepithelium and surrounding mesenchymal cells, indicating that the epidermal growth factor plays an important regulatory role in development and differentiation of embryonic neural stem cells in vivo. In some embodiments, the working concentration of the epidermal growth factor is 1-500 ng/ml, preferably 5-100 ng/ml, further preferably 10-30 ng/ml. The above working concentrations are concentrations of various cytokines and/or cytokine inhibitors in culture media.

In some embodiments, the exogenous activator can be replaced with an endogenous activator, and the non-limiting example of the endogenous activator is microRNA. The sequence of microRNA is highly conservative between many cellular biological species, which can participate in many important biological events including cell proliferation, differentiation, apoptosis, metabolism, and stress response and the like. In the present disclosure, microRNA affects the endogenous expression of the NFIX gene by activating or interfering with their upstream and downstream pathways. The non-limiting examples of microRNA include miR21, miR181b and miR153.

In some embodiments, the glial cell mutation promoting factor is one or more selected from the group consisting of: a leukocyte inhibitory factor, fetal bovine serum, newborn bovine serum, an adult bovine serum and sheep serum and their analogues, a BMP activator, a neurotrophic factor and/or other reported glial cell mutation promoting factors. The leukocyte inhibitory factor (LIF), fetal bovine serum, newborn bovine serum, adult bovine serum and sheep serum and their analogues, BMP activator and neurotrophic factor can effectively promote the differentiation of glial precursor cells into glial cells. In some embodiments, the glial cell mutation promoting factor is a combination of at least one of the above serums and the leukocyte inhibitory factor. In some embodiments, the glial cell mutation promoting factor is at least one of the above serums. In some embodiments, the glial cell mutation promoting factor is the leukocyte inhibitory factor. In some embodiments, the working concentration of the leukocyte inhibitory factor is 1-200 ng/ml, preferably 5-100 ng/ml, further preferably 10-30 ng/ml. In some embodiments, the working concentration of the serum is 1%-50%, preferably 2%-20%, further preferably 5%-10%; and the percentage is volume percentage. The above working concentrations are concentrations of various cytokines and/or cytokine inhibitors in culture media.

In some embodiments, oligodendrocytes are obtained by induction. The cytokine added in step 2) is one or more selected from the group consisting of: retinoic acid (RA), fibroblast growth factor (FGF, such as FGF2), platelet derived growth factor (PDGF), insulin-like growth factor (IGF, such as IGF-1), neurotrophin 3 (NT3), ventral morphogenetic hormone SHH, purmorphamine, and Hedgehog agonist SAG.

In some embodiments, microglia are obtained by induction, and the cytokine added in step 2) is one or more selected from the group consisting of: fibroblast growth factor (FGF, such as FGF2), stem cytokine (SCF), vascular endothelial growth factor (VEGF), interleukin (IL, such as IL-3, IL-34, etc.), thrombopoietin, macrophage colony-stimulating factor (M-CSF), FMS like tyrosine kinase 3 ligand (Flt3l), granulocyte macrophage colony stimulating factor (GM-CSF), or GlutaMAX-I.

In some embodiments, the method comprises the following steps:
(1) constructing positive cloned human stem cells that overexpresses a reprogramming factor through a CRISPR/Cas9 system, wherein the reprogramming factor comprises an NFIX gene;
(2) inducing the positive cloned human stem cells into the neural precursor cells by adding a TGF-β inhibitor and a BMP inhibitor;
(3) inducing the neural precursor cells into glial precursor cells by adding a neural differentiation promoting factor (such as EGF and/or FGF); and
(4) inducing the glial precursor cells into glial cells by adding a glial cell mutation promoting factor.

In some embodiments, the glial cells obtained by the method are astrocytes.

In some embodiments, the method does not comprise a step of purifying the glial cells. The existing induction method is poor in cell maturity and many in parenchyma cells due to low induction efficiency. The existence of parenchyma cells can further affect the differentiation direction of cells, and therefore purification is needed. However, in the method of the present disclosure, the cell population obtained by overexpression of NFIX genes is high in differentiation efficiency and good in maturity, even without purification steps (such as sorting) to remove undifferentiated cells, it can maintain the survival and development of neurons and has good druggability.

1.5 Further Obtaining of Brain and/or Spinal Cord Specialized Subtype Glial Cells

In some embodiments, provided is a method for obtaining brain and/or spinal cord specialized subtype glial cells in vitro, on the basis of the above steps 1) and 2), further comprising a step 3): inducing the glial cells to brain and/or spinal cord specialized subtype glial cells by adding at least one of other cytokines and/or cytokine inducers.

In some embodiments, the brain and/or spinal cord specialized subtype glial cells are one or more selected from the group consisting of: forebrain specialized subtype glial cells, midbrain specialized subtype glial cells, back brain specialized subtype glial cells, and different spinal cord segment specialized subtype glial cells. By taking astrocytes as an example, they are one or more selected from the group consisting of: forebrain specialized astrocytes, midbrain specialized astrocytes, back brain specialized astrocytes, and different spinal cord segment specialized astrocytes. By taking oligodendrocytes as an example, they are one or more selected from the group consisting of: forebrain specialized oligodendrocytes, midbrain specialized oligodendrocytes, back brain specialized oligodendrocytes, and different spinal cord segment specialized oligodendrocytes. By taking microglias as an example, they are one or more selected from the group consisting of: forebrain specialized microglias, midbrain specialized microglias, back brain specialized microglias, and different spinal cord segment specialized microglias.

In some embodiments, the other cytokines and/or cytokine inducers are added in step 3). The other cytokines and/or cytokine inducers are known by those skilled in the art, can be used as conventional reagents for inducing regional specialization of glial cells, such as brain specialization and spinal cord specialization. As known by those skilled in the art, when morphogens are added, the cells can be further induced to dorsal forebrain glial cells; when retinoic acid (RA) is added, the cells can be further induce to spinal cord glial cells; when the ventral morphogenetic hormone SHH is added, the cells can be further induced to ventral forebrain glial cells.

2. Glial Cells and Drugs Comprising the Same

According to another aspect of the present disclosure, provided are glial cells obtained by the above method.

In some embodiments, the glial cells are one or more selected from the group consisting of astrocytes, oligodendrocytes, microglias and other types of glial cells, and the combination thereof.

In some embodiments, the glial cells are astrocytes.

In some embodiments, provided are brain and/or spinal cord specialized subtype glial cells obtained by the above method.

In some embodiments, provided is a glial cell population obtained by the above method.

In some embodiments, the glial cell population is one or more selected from the group consisting of an astrocyte population, an oligodendrocyte population, a microglia population, other types of glial population and the combination thereof.

In some embodiments, the glial cell population is the astrocyte population.

In some embodiments, the above astrocyte population can be obtained by the above method without the purification process. In the astrocyte population, the quantity percentage of the astrocyte population is at least above 50%, at least above 60%, at least above 70%, and at least above 80%.

In some embodiments, provided is a brain and/or spinal cord specialized subtype glial cell population.

In some embodiments, the above cell population is obtained by the above method without the purification process, such as sorting. In the cell population, the quantity percentage of the target cells (astrocytes, oligodendrocytes, microglias, and brain and/or spinal cord specialized subtype glial cells) is at least above 50%, at least above 60%, at least above 70% and at least above 80%.

Compared with cells obtained in the prior art, the cells obtained by the present disclosure are higher in differentiation efficiency, more in protrusion, bigger in cells and better in maturity. Even though the cells are directly used without purification, they have good support and nutrition effects on neurons. The therapeutic drugs prepared from the cells of the present disclosure have good application prospect.

According to another aspect of the present disclosure, provided is a drug, comprising the glial cells obtained by the above method.

In some embodiments, provided is a drug, comprising the astrocytes obtained by the above method.

In some embodiments, the drug comprises a) the glial cells obtained by the above method; and/or b) the brain and/or spinal cord specialized subtype glial cells obtained by the above method.

In some embodiments, provided is a drug, comprising the glial cell population obtained by the above method.

In some embodiments, the drug comprises d) the glial cell population obtained by the above method; and/or e) the brain and/or spinal cord specialized subtype glial cell population obtained by the above method.

In some embodiments, the drug comprises the astrocyte population obtained by the above method.

In some embodiments, the drug is a cell treatment drug.

In some embodiments, the drug further comprises at least one pharmaceutically acceptable carrier. The various desired dosage forms can be prepared by adding suitable carriers.

3. Therapeutic Method

According to another aspect of the present application, provided is use of the above cells or cell population in the preparation of a drug for preventing and/or treating a nervous system disease.

In some embodiments, the nervous system disease is a neurodegenerative disease. In some embodiments, the neurodegenerative disease is at least one selected from the group consisting of Alzheimer's disease, amyotrophic lateral sclerosis, Parkinson's disease, schizophrenia, glioblastoma, Huntington's disease, multiple sclerosis and the like.

According to another aspect of the present disclosure, provided is a method for preventing and/or treating a nervous system disease, comprising: administrating to a subject an effective amount of at least one of: a) the glial cells; b) the brain and/or spinal cord specialized subtype glial cells; and/or c) a drug including the a) and/or b). The cells or drugs can be systemically or locally administered to the subject. The cells or drugs can be administrated (such as injection) to a target organ. For example, the cells or drugs can be administrated to any part of a subject comprising effective nerves, including but not limiting to brain.

According to another aspect of the present disclosure, provided is a method for preventing and/or treating a nervous system disease, comprising: administrating to a subject an effective amount of at least one of: d) the glial cell population; e) the brain and/or spinal cord specialized subtype glial cell population; and/or c) a drug including the d) and/or e). The cell populations or drugs can be systemically or locally administered to the subject. The cell populations or drugs can be administrated (such as injection) to a target organ. For example, the cell populations or drugs can be administrated to any part of a subject comprising effective nerves, including but not limiting to brain.

4. Drug Screening Kit and Method

According to another aspect of the present disclosure, provided is an in-vitro or in-vivo drug screening kit, comprising glial cells obtained by the above method.

According to another aspect of the present disclosure, provided is an in-vitro or in-vivo drug screening kit, comprising a) glial cells; and/or b) brain and/or spinal cord specialized subtype glial cells.

According to another aspect of the present disclosure, provided is an in-vitro or in-vivo drug screening kit, comprising d) a glial cell population; and/or e) a brain and/or spinal cord specialized subtype glial cell population.

According to another aspect of the present disclosure, provided is an in-vitro drug screening method.

In some embodiments, a using method of the screening kit or the screening method comprises: contacting a) glial cells and/or b) brain and/or spinal cord specialized subtype glial cells with a test compound; and detecting changes in cell morphology, biomarkers and functional activity, so as to determine whether the compound can prevent and/or treat neurodegenerative diseases.

In some embodiments, a using method of the screening kit or the screening method comprises: contacting d) a glial cell population and/or e) a brain and/or spinal cord specialized subtype glial cell population with a test compound; and detecting changes in cell morphology, biomarkers and functional activity, so as to determine whether the compound can prevent and/or treat neurodegenerative diseases.

5. Induction Kit

According to another aspect of the present disclosure, provided is a kit for inducing the differentiation of stem cells, comprising:
1) at least one reprogramming factor overexpression reagent, wherein the reprogramming factor comprises an NFIX gene; and
2) at least one cytokine and/or cytokine inhibitor.

In some embodiments, the cytokine and/or the cytokine inhibitor is one or more selected from the group consisting of: an ectoderm and neural differentiation promoting factor and/or a non-neural differentiation inhibitor, a neural differentiation promoting factor, a glial cell maturation promoting factor, and other reported cytokines and/or cytokine inhibitors that are capable of inducing positive cloned stem cells to glial cells. One or more (i.e., alone or a combination) of the above cytokines and/or cytokine inhibitors are capable of inducing the positive cloned stem cells to the glial cells. In some embodiments, the ectodermal and neural differentiation promoting factor and/or non-neural differentiation promoting inhibitor is a transforming growth factor inhibitor. In some embodiments, the transforming growth factor inhibitor is a TGF-β inhibitor and/or a BMP inhibitor. In some embodiments, the neural differentiation promoting factor is an exogenous activator. The non-limiting examples of the exogenous activators include fibroblast growth factors and/or epidermal growth factors and/or small molecule functional analogues and/or other functional analogues. In some embodiments, the exogenous activator can be replaced with an endogenous activator which may be microRNA. In some embodiments, the glial cell mutation promoting factor is one or more selected from the group consisting of: a leukocyte inhibitory factor, a fetal bovine serum, a newborn bovine serum, an adult bovine serum and sheep serum and their analogues, a BMP activator, a neurotrophic factor and/or other reported glial cell mutation promoting factors. The leukocyte inhibitory factor (LIF), fetal bovine serum, newborn bovine serum, adult bovine serum and sheep serum and their analogues, BMP activator and neurotrophic factor can effectively promote the differentiation of glial precursor cells into glial cells.

In some embodiments, the at least one cytokine and/or cytokine inhibitor in the step 2) comprises: a) a TGF-β inhibitor and/or a BMP inhibitor; and b) EGF and/or FGF; and c) the glial cell mutation promoting factor which is one or more selected from the group consisting of: a leukocyte inhibitory factor, a fetal bovine serum, a newborn bovine serum, an adult bovine serum and sheep serum and their analogues, a BMP activator, a neurotrophic factor and/or other reported glial cell mutation promoting factors.

In some embodiments, the kit further comprises: 3) at least one of other cytokines and/or cytokine inducers which are used for further inducing the glial cells into the brain and/or spinal cord specialized subtype glial cells.

In some embodiments, the kit further comprises: stem cells. In some embodiments, the kit further comprises: an instruction for directed induction of stem cells into glial cells; and/or an instruction for further induced differentiation of the glial cells into brain and/or spinal cord specialized subtype glial cells.

Next, the beneficial effects of the present application will be further illustrated.

### Example 1

A human embryonic stem cell line (H9, with a passage number of 20-40, derived from the WiCell cell bank in the United States) was cultured in E8 culture medium, and NFIX was transfected on the human pluripotent stem cell line through a CRISPR/Cas9 system to overexpress. The cells were digested with Dispase (1 mg/ml, Gibco/Thermo) when the confluence was 60%-80%, and spread onto a 6-well culture plate.

On the next day, a first-stage culture medium: DMEM/DF12+N2 (1%) (Neurobasal culture medium) (Thermo)+SB431542 (2 μM) (R&D/PeproTech)+LDN193189 (100 nM) (R&D/PeproTech), was added for inducing for 10 days.

After 10 days of induction, the cells were digested for 3 minutes with ethylene diamine tetraacetic acid (EDTA), and cultured in a suspension culture flask containing a second-stage culture medium: Neurobasal culture medium+FGF2 (10 ng/ml) (R&D/PeproTech)+EGF (10 ng/ml) (R&D/PeproTech), to induce for 25 days.

After 25 days of induction, the cells were digested with Dispase (1 mg/ml, Gibco/Thermo) and spread onto a 6-well culture plate, and added with a third-stage culture medium: Neurobasal culture medium+LIF (10 ng/ml) (R&D/PeproTech), to induce for 7 days, so that rapidly induced human glial cells were obtained.

### Example 2

A human embryonic stem cell line (H9, with a passage number of 20-40, derived from the WiCell cell bank in the United States) was cultured in E8 culture medium, and NFIX was transfected on the human pluripotent stem cell line through a CRISPR/Cas9 system to overexpress. The cells were digested with Dispase (1 mg/ml, Gibco/Thermo) when the confluence was 60%-80%, and spread onto a 6-well culture plate.

On the next day, a first-stage culture medium: DMEM/DF12+N2 (1%) (Neurobasal culture medium) (Thermo)+SB431542 (2 μM) (R&D/PeproTech)+LDN193189 (100 nM) (R&D/PeproTech), was added for inducing for 10 days.

After 10 days of induction, the cells were digested for 3 minutes with EDTA, and cultured in a suspension culture flask containing a second-stage culture medium: Neurobasal culture medium+FGF2 (10 ng/ml) (R&D/PeproTech)+EGF (10 ng/ml) (R&D/PeproTech), to induce for 21 days.

After 21 days of induction, the cells were digested with Dispase (1 mg/ml, Gibco/Thermo) and spread onto a 6-well culture plate, and added with a third-stage culture medium: neurobasal culture medium with 5% fetal bovine serum+LIF (10 ng/ml) (R&D/PeproTech), to induce for 7 days, so that rapidly induced human glial cells were obtained.

### Example 3

A human embryonic stem cell line (H9, with a passage number of 20-40, derived from the WiCell cell bank in the United States) was cultured in E8 culture medium, and NFIX was transfected on the human pluripotent stem cell line through a CRISPR/Cas9 system to overexpress. The cells were digested with Dispase (1 mg/ml, Gibco/Thermo) when the confluence was 60%-80%, and spread onto a 6-well culture plate.

On the next day, a first-stage culture medium: DMEM/DF12+N2 (1%) (Neurobasal culture medium) (Thermo)+SB431542 (2 μM) (R&D/PeproTech)+LDN193189 (100 nM) (R&D/PeproTech), was added for inducing for 7 days.

After 7 days of induction, the cells were digested for 3 minutes with EDTA, and cultured in a suspension culture flask containing a second-stage culture medium: Neurobasal culture medium+FGF2 (10 ng/ml) (R&D/PeproTech)+EGF (10 ng/ml) (R&D/PeproTech), to induce for 28 days.

After 28 days of induction, the cells were digested with Dispase (1 mg/ml, Gibco/Thermo) and spread onto a 6-well culture plate, and added with a third-stage culture medium: neurobasal culture medium with 5% fetal bovine serum+LIF (10 ng/ml) (R&D/PeproTech), to induce for 7 days, so that rapidly induced human glial cells were obtained.

### Example 4

A human embryonic stem cell line (H1, with a passage number of 20-40, derived from the WiCell cell bank in the United States) was cultured in E8 culture medium, and NFIX was transfected on the human pluripotent stem cell line through a CRISPR/Cas9 system to overexpress. The cells were digested with Dispase (1 mg/ml, Gibco/Thermo) when the confluence was 60%-80%, and spread onto a 6-well culture plate.

On the next day, a first-stage culture medium: DMEM/DF12+N2 (1%) (Neurobasal culture medium) (Thermo)+SB431542 (2 μM) (R&D/PeproTech)+LDN193189 (100 nM) (R&D/PeproTech), was added for inducing for 10 days.

After 10 days of induction, the cells were digested for 3 minutes with EDTA, and cultured in a suspension culture flask containing a second-stage culture medium: Neurobasal culture medium+FGF2 (10 ng/ml) (R&D/PeproTech)+EGF (10 ng/ml) (R&D/PeproTech), to induce for 25 days.

After 25 days of induction, the cells were digested with Dispase (1 mg/ml, Gibco/Thermo) and spread onto a 6-well culture plate, and added with a third-stage culture medium: neurobasal culture medium with 10% fetal bovine serum, to induce for 7 days, so that rapidly induced human glial cells were obtained.

### Example 5

An induced human pluripotent stem cell (WC50, derived from the WiCell cell bank in the United States) was cultured in E8 culture medium, and NFIX was transfected on the human pluripotent stem cell line through a CRISPR/Cas9 system to overexpress. The cells were digested with Dispase (1 mg/ml, Gibco/Thermo) when the confluence was 60%-80%, and spread onto a 6-well culture plate.

On the next day, a first-stage culture medium: DMEM/DF12+N2 (1%) (Neurobasal culture medium) (Thermo)+SB431542 (2 μM) (R&D/PeproTech)+LDN193189 (100 nM) (R&D/PeproTech), was added for inducing for 10 days.

After 10 days of induction, the cells were digested for 3 minutes with EDTA, and cultured in a suspension culture flask containing a second-stage culture medium: Neurobasal culture medium+FGF2 (10 ng/ml) (R&D/PeproTech)+EGF (10 ng/ml) (R&D/PeproTech), to induce for 22 days.

After 22 days of induction, the cells were digested with Dispase (1 mg/ml, Gibco/Thermo) and spread onto a 6-well culture plate, and added with a third-stage culture medium: Neurobasal culture medium+LIF (10 ng/ml) (R&D/PeproTech), to induce for 7 days, so that rapidly induced human glial cells were obtained.

### Example 6

An induced human pluripotent stem cell (WC50, derived from the WiCell cell bank in the United States) was cultured in E8 culture medium, and NFIX was transfected on the human pluripotent stem cell line through a CRISPR/Cas9 system to overexpress. The cells were digested with Dispase (1 mg/ml, Gibco/Thermo) when the confluence was 60%-80%, and spread onto a 6-well culture plate.

On the next day, a first-stage culture medium: DMEM/DF12+N2 (1%) (Neurobasal culture medium) (Thermo)+SB431542 (2 μM) (R&D/PeproTech)+LDN193189 (100 nM) (R&D/PeproTech), was added for inducing for 10 days.

After 10 days of induction, the cells were digested for 3 minutes with EDTA, and cultured in a suspension culture flask containing a second-stage culture medium: Neurobasal culture medium+FGF2 (10 ng/ml) (R&D/PeproTech)+EGF (10 ng/ml) (R&D/PeproTech), to induce for 26 days.

After 26 days of induction, the cells were digested with Dispase (1 mg/ml, Gibco/Thermo) and spread onto a 6-well culture plate, and added with a third-stage culture medium: Neurobasal culture medium with 5% fetal bovine serum+LIF (10 ng/ml) (R&D/PeproTech), to induce for 7 days, so that rapidly induced human glial cells were obtained.

### Example 7

A human embryonic stem cell line (H9, with a passage number of 20-40, derived from the WiCell cell bank in the United States) was cultured in E8 culture medium, and NFIX was transfected on the human pluripotent stem cell line through a CRISPR/Cas9 system to overexpress. The cells were digested with Dispase (1 mg/ml, Gibco/Thermo) when the confluence was 60%-80%, and spread onto a 6-well culture plate.

On the next day, a first-stage culture medium: DMEM/DF12+N2 (1%) (Neurobasal culture medium) (Thermo)+SB431542 (2 μM) (R&D/PeproTech)+LDN193189 (100 nM) (R&D/PeproTech), was added for inducing for 10 days.

After 10 days of induction, the cells were digested for 3 minutes with EDTA, and cultured in a suspension culture flask containing a second-stage culture medium: Neurobasal culture medium+FGF2 (10 ng/ml) (R&D/PeproTech)+EGF (10 ng/ml) (R&D/PeproTech), to induce for 25 days.

After 25 days of induction, the cells were digested with Dispase (1 mg/ml, Gibco/Thermo) and spread onto a 6-well culture plate, and added with a third-stage culture medium: Neurobasal culture medium+LIF (10 ng/ml) (R&D/PeproTech), to induce for 7 days, so that rapidly induced human glial cells were obtained.

After maturation promoting induction for 7 days, the obtained human glial cells were digested using Dispase (1 mg/ml, Gibco/Thermo) and spread onto a 6-well culture plate, and added with the Neurobasal culture medium for monolayer culture. On the next day, the obtained human glial cells and the neurons differentiated by the human pluripotent stem cells with strong GFP expression were spread at a ratio of 1:1 for co-culture for 7 days, then the promotion and support effect of the human pluripotent stem cells obtained by induction on the neurons was analyzed.

### Comparative Example 1

A human embryonic stem cell line (H9, with a passage number of 20-40, derived from the WiCell cell bank in the United States) was cultured in E8 culture medium, digested with Dispase (1 mg/ml, Gibco/Thermo) when the confluence was 60%-80%, and spread onto a 6-well culture plate.

### Comparative Example 2

A human embryonic stem cell line (H9, with a passage number of 20-40, derived from the WiCell cell bank in the United States) was cultured in E8 culture medium, digested with Dispase (1 mg/ml, Gibco/Thermo) when the confluence was 60%-80%, and spread onto a 6-well culture plate.

On the next day, a first-stage culture medium: DMEM/DF12+N2 (1%) (Neurobasal culture medium)+SB431542 (2 μM) (R&D/PeproTech)+LDN193189 (100 nM) (R&D/PeproTech), was added for inducing for 10 days.

After 10 days of induction, the cells were digested for 3 minutes with EDTA, cultured in a suspension culture flask containing a second-stage culture medium: Neurobasal culture medium+FGF2 (10 ng/ml) (R&D/PeproTech)+EGF (10 ng/ml) (R&D/PeproTech), to induce for 25 days.

After 25 days of induction, the cells were digested with Dispase (1 mg/ml, Gibco/Thermo) and spread onto a 6-well culture medium, and added with a third-stage culture medium: Neurobasal culture medium+LIF (10 ng/ml) (R&D/PeproTech), to induce for 7 days, so that early differentiated cells which were not fully specialized into glial cells were obtained.

### Comparative Example 3

A human embryonic stem cell line (H9, with a passage number of 20-40, derived from the WiCell cell bank in the United States) was cultured in E8 culture medium, and NFIA was transfected on the human pluripotent stem cell line through a CRISPR/Cas9 system to overexpress. The cells were digested with Dispase (1 mg/ml, Gibco/Thermo) when the confluence was 60%-80%, and spread onto a 6-well culture plate.

On the next day, a first-stage culture medium: DMEM/DF12+N2 (1%) (Neurobasal culture medium) (Thermo)+SB431542 (2 μM) (R&D/PeproTech)+LDN193189 (100 nM) (R&D/PeproTech), was added for inducing for 10 days.

After 10 days of induction, the cells were digested for 3 minutes with EDTA, and cultured in a suspension culture flask containing a second-stage culture medium: Neurobasal culture medium+FGF2 (10 ng/ml) (R&D/PeproTech)+EGF (10 ng/ml) (R&D/PeproTech), to induce for 25 days.

After 25 days of induction, the cells were digested with Dispase (1 mg/ml, Gibco/Thermo) and spread onto a 6-well culture plate, and added with a third-stage culture medium: Neurobasal culture medium+LIF (10 ng/ml) (R&D/PeproTech), to induce for 7 days, so that rapidly induced human glial cells were obtained.

### Result Analysis:

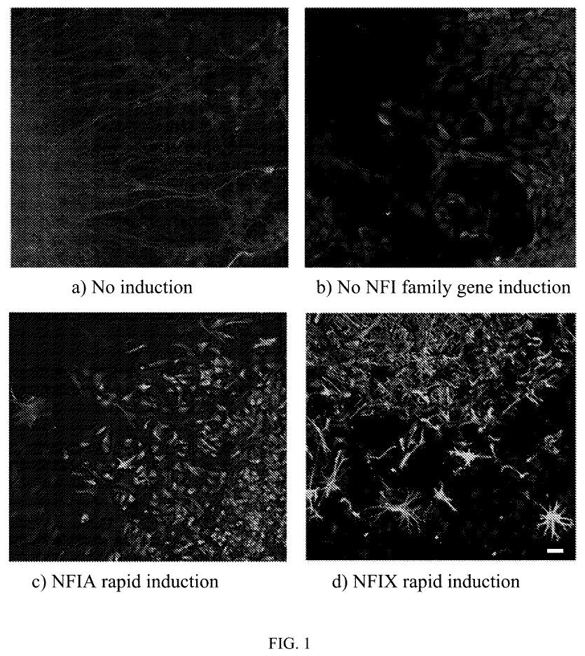

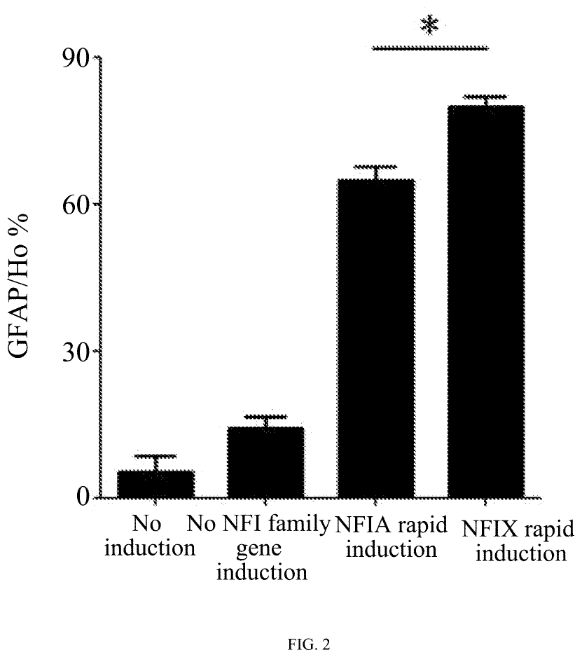

1. The Nikon laser confocal microscope was used to observe cell fluorescence display results of Example 1, Comparative example 1, Comparative example 2 and Comparative example 3 under 488 nm of excitation light and under the same appropriate exposure time. The results are shown in FIG. 1 and FIG. 2, where GFAP: glial fibrillary acidic protein staining, used for staining astrocytes; Ho: Hochester staining, used for staining all cells, including successfully induced and non-induced cells. The test method was Student's t-test, and data was expressed as mean+/−SEM, *P<0.05; **P<0.01; ***P<0.001; ****P<0.0001. FIG. 1 (scale=100 μm) and FIG. 2 show that without induction, pluripotent stem cells only spontaneously differentiate into a very low proportion of weakly positive GFAP⁺ cells that do not possess glial cell morphological characteristics, and such cells are not considered to have been specialized into glial cells (Comparative example 1); without the induction of NFI family genes, the expression level of the marker gene GFAP of glial cells is low or the marker gene GFAP of glial cells is not expressed, and the induction efficiency is low (Comparative example 2); by using NFIA rapid induction (Comparative example 3), GFAP and glial cells with high induction efficiency can be obtained, and the induced cells have primary maturity, which is manifested in low gene expression, low complexity of neurites and star shaped neurites, small number of neurites, and small cells; and by using NFIX rapid induction (Example 1), GFAP and glial cells with significantly improved induction efficiency and the highest induction efficiency under parallel contrast conditions can be obtained, and the induced cells have high maturity, which is manifested in high gene expression, high complexity of neurites and star shaped neurites, large number of neurites and large cells. FIG. 2 show that even compared to the NFIA rapid induction group (approximately 65%), the GFAP/Ho % (approximately 83%) of the NFIX rapid induction group is significantly higher (P<0.05), indicating a higher differentiation efficiency in the NFIX rapid induction group.

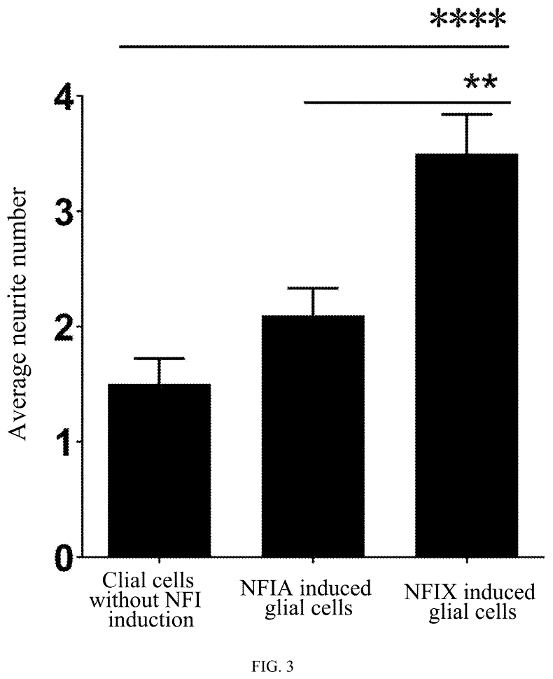

The number of neurites in Example 1 was compared with that in Comparative example 2 and Comparative example 3, and the results are shown in FIG. 3. The results show that the number of neurites of glial cells rapidly induced by NFIX is extremely significant more than that of glial cells without the induction of NFI family genes (P<0.0001), and also significantly more than that of glial cells rapidly induced by NFIA (P<0.01, specifically, P=0.0031), further proving that the maturity of glial cells obtained by NFIX rapidly induction group is better.

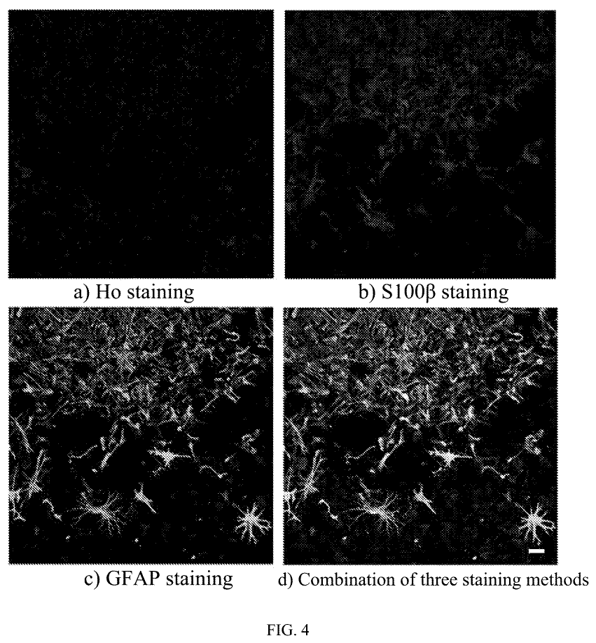

2. The glial cells obtained from Example 1 were subjected to glial cells (astrocytes) S100beta (S100 β) staining and Hochester staining, the staining of the two specific marker genes (S100beta and GFAP) confirms that the cells obtained by induction are human glial cells, as shown in FIG. 4 (scale=100 μm).

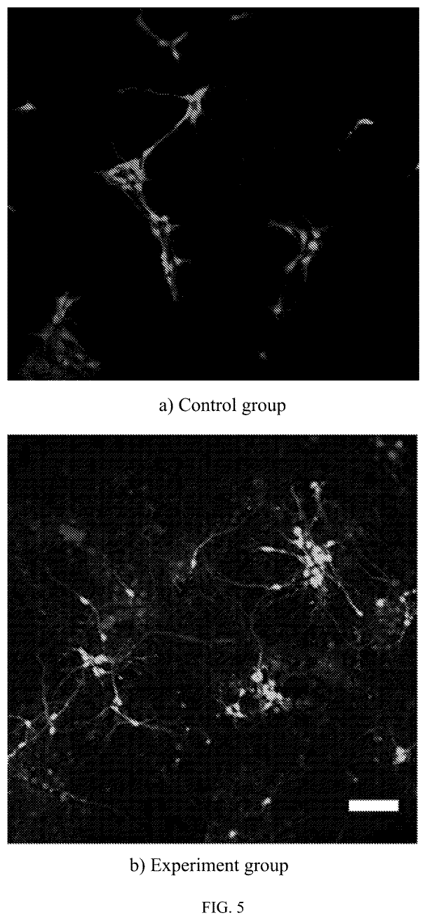

3. The results from Example 7 show that exogenous GFP strong-expression in-vivo labeled neuron cells were co-cultured with the glial cells rapidly induced by NFIX. The results are shown in FIG. 5 (scale=100 μm). In FIG. 5, a) shows neurons differentiated from human pluripotent stem cells strongly expressing GFP (i.e., control group), and b) shows 7-day co-culture with glial cells obtained from Example 1 (i.e., experimental group). As can be seen, the glial cells rapidly induced by NFIX can well maintain the survival of neurons and the development and extension of neurites.

It can be seen that in the present disclosure, the NFIX gene is used to directly induce human stem cells, so as to obtain a sufficient number of high-quality seed cells. Owing to its infinite passage advantage, the process and time costs are directly reduced, and the batch stability is enhanced. Compared to the traditional induction methods, duce to the high differentiation induction efficiency of NFIX, the method of present disclosure greatly shortens the required time and directly reduces the induction cost. That is, in the present disclosure, highly matured glial cells can be obtained through rapid and efficient induction.

Furthermore, the inventor unexpectedly found that even compared to the family gene NFIA, the NFIX rapid induction group had significantly better differentiation efficiency and cell maturity; in terms of induction quantity, the NFIX rapid induction group was able to obtain more glial cells with a lower proportion of parenchyma cells, thereby reducing the indirect cost of subsequent sorting steps; in terms of induction quality, the expression level of marker genes was higher, resulting in larger cells, more protrusions, and higher complexity of protrusions and star structures. Further, in combination with the staining identification of target cells and the relevant functional results, it can be seen that the glial cells obtained by the present disclosure have a supportive and promoting effect on the morphology and function of neurons, and are significantly superior in maturity and performance to the glial cells obtained in existing technologies, which have better application prospects and lower application costs in the treatment of diseases of the nervous system and in drug screening.

The above description provides a detailed introduction to the embodiments of the present application. Specific examples are used to explain the principles and implementation methods of this application. The explanations of the above embodiments are only used to help understand the methods and core ideas of this application. Meanwhile, any changes or deformations made by those in the art based on the specific implementation method and application scope of this application, in accordance with the ideas of this application, are within the scope of protection of this application. In conclusion, the content of the present description should not be understood as limiting the present application.

1. A method for obtaining glial cells in vitro, comprising:
1) constructing positive cloned stem cells that overexpress a reprogramming factor, wherein the reprogramming factor comprises an NFIX gene; and
2) inducing the positive cloned stem cells to the glial cells by adding a cytokine and/or a cytokine inhibitor.

2. The method according to claim 1, wherein the positive cloned stem cells are constructed through a CRISPR/Cas9 system.

3. The method according to claim 1, wherein the cytokine and/or the cytokine inhibitor in the step 2) are/is one or more of: an ectodermal and neural differentiation promoting factor and/or a non-neural differentiation promoting inhibitor, a neural differentiation promoting factor, or a glial cell maturation promoting factor.

4. The method according to claim 1, wherein the stem cells are pluripotent stem cells and/or neural stem cells; preferably, the pluripotent stem cells are embryonic stem cells and/or induced pluripotent stem cells.

5. The method according to claim 1, wherein the reprogramming factor further comprises at least one of other genes that are beneficial for directed differentiation into the glial cells.

6. The method according to claim 1, wherein the reprogramming factor further comprises at least one of other nuclear factor genes in NFI family genes excluding NFIX; preferably, the other nuclear factor genes are NFIA and/or NFIB.

7. The method according to claim 3, wherein the ectodermal and neural differentiation promoting factor and/or non-neural differentiation promoting inhibitor are/is a transforming growth factor inhibitor; preferably, the transforming growth factor inhibitor is a TGF-β inhibitor and/or a BMP inhibitor.

8. The method according to claim 3, wherein the neural differentiation promoting factor is an exogenous activator; preferably, the exogenous activator is a fibroblast growth factor and/or an epidermal growth factor.

9. The method according to claim 3, wherein the glial cell maturation promoting factor is one or more selected from the group consisting of: a leukocyte inhibitory factor, a fetal bovine serum, a newborn bovine serum, an adult bovine serum and sheep serum, a BMP activator and a neurotrophic factor.

10. The method according to claim 3, wherein the induction comprises three stage cultivation, the ectodermal and neural differentiation promoting factor and/or the non-neural differentiation inhibitor are/is added in the first stage, the neural differentiation promoting factor is added in the second stage, and the glial cell maturation promoting factor is added in the third stage.

11. The method according to claim 1, further comprising a step 3): inducing the glial cells to brain and/or spinal cord specialized subtype glial cells by adding at least one of other cytokines and/or cytokine inducers.

12. Glial cells obtained by the method according to claim 1.

13. Brain and/or spinal cord specialized subtype glial cells obtained by the method according to claim 11.

14. A drug, comprising the glial cells according to claim 12.

15. A drug, comprising a) the glial cells obtained by a method comprising:
1) constructing positive cloned stem cells that overexpress a reprogramming factor, wherein the reprogramming factor comprises an NFIX gene; and
2) inducing the positive cloned stem cells into the glial cells by adding a cytokine and/or a cytokine inhibitor;
and/or b) the brain and/or spinal cord specialized subtype glial cells according to claim 13.

16. A method for preventing and/or treating a nervous system disease comprising: administrating the glial cells according to claim 12 to a subject in need thereof.

17. A method for preventing and/or treating a nervous system disease comprising: administrating the brain and/or spinal cord specialized subtype glial cells according to claim 13 to a subject in need thereof.

18. An in-vitro or in-vivo drug screening kit, comprising a) the glial cells obtained by a method comprising:
1) constructing positive cloned stem cells that overexpress a reprogramming factor, wherein the reprogramming factor comprises an NFIX gene; and
2) inducing the positive cloned stem cells into the glial cells by adding a cytokine and/or a cytokine inhibitor;
and/or b) the brain and/or spinal cord specialized subtype glial cells according to claim 13.

19. A kit for inducing the differentiation of stem cells, comprising:
1) at least one reprogramming factor overexpression reagent, wherein the reprogramming factor comprises an NFIX gene; and
2) at least one cytokine and/or cytokine inhibitor for inducing the positive cloned stem cells that overexpress the reprogramming factor into the glial cells.

20. The kit according to claim 19, further comprising: 3) at least one of other cytokines and/or cytokine inducers for further inducing the glial cells into the brain and/or spinal cord specialized subtype glial cells.
